(19) United States
(12) Patent Application Publication
(10) Pub. No.: US 2025/0143587 A1
(43) Pub. Date: May 8, 2025

(54) SYSTEMS AND METHODS FOR DETERMINING A VISCOSITY OF A FLUID

(71) Applicant: KARDION GMBH, Stuttgart (DE)

(72) Inventors: Ingo Stotz, Ditzingen (DE); Thomas Alexander Schlebusch, Renningen (DE)

(21) Appl. No.: 18/942,153

(22) Filed: Nov. 8, 2024

Related U.S. Application Data
(63) Continuation of application No. 15/734,519, filed on Jul. 20, 2021, now Pat. No. 12,178,554.

(30) Foreign Application Priority Data
Jun. 6, 2018 (DE) 102018208936.1

(51) Int. Cl. A61B 5/02; A61B 5/00; A61B 5/0215; A61B 8/00; A61M 60/178; A61M 60/216; A61M 60/403; A61M 60/523; A61M 60/531

(52) U.S. Cl. CPC A61B 5/02035; A61B 5/0215; A61B 5/7246; A61B 8/488; A61M 60/178; A61M 60/216; A61M 60/403; A61M 60/523; A61M 60/531; A61M 2205/3331; A61M 2205/3365; A61M 2205/3375; A61M 2205/3379

(57) ABSTRACT

The approach presented here relates to a determination appliance (100) for determining a viscosity of a fluid. The determination appliance (100) has at least one determination device (110) and a provisioning device (115). The determination device (110) is designed to determine the viscosity of the fluid and/or a rotational speed (ω) of a blade wheel (205) for conveying the fluid by using at least one detected volume flow of the fluid and a detected pressure difference of the fluid. The provisioning device (115) is designed to provide or send a viscosity signal (130) representing the viscosity determined by the determination device (110).

The invention relates to a determination appliance and to a method for determining a viscosity of a fluid. The invention also relates to a computer program and to a machine-readable storage medium on which the computer program is stored.

PT (prothrombin time) and INR (international normalized ratio) are the standard measure for blood coagulation. Usually, the INR in blood samples is determined by adding thromboplastin and subsequently measuring the time to coagulation. The determination can take place in the laboratory; today, test strip devices are also available for self-measurement by the patient, comparable to the procedure of a blood sugar measurement. For patients with heart support systems, so-called coagulation management is essential for minimizing pump thromboses. Monitoring of blood viscosity as an INR replacement parameter may be sufficient for coagulation management.

EP 2 175 770 B1 describes an explicit blood viscosity sensor on the basis of surface acoustic waves, SAW for short, for determining viscosity.

U.S. Pat. No. 7,591,777 B2 describes a viscosity determination in heart support systems by means of the mechanical effect of the blood viscosity on the drive of the heart support system.

The object of the invention is to specify an improved method for determining a viscosity of a fluid and an improved determination appliance for this purpose. It is in particular an object of the invention to specify a method and an appliance that allows the continuous determination of the viscosity of a fluid on a short time scale.

This object is achieved by the determination appliance specified in claim 1 and the method specified in claim 9. Advantageous embodiments of the invention are specified in the dependent claims.

In the following, a determination appliance for determining a viscosity of a fluid and a method according to the invention for determining a viscosity of a fluid and, lastly, a corresponding computer program are presented. Advantageous developments and improvements of the objects specified in the independent claims are possible by means of the measures listed in the dependent claims.

The advantages achievable with the presented approach are that a determination appliance presented here is designed to quickly and easily determine and provide or send the viscosity of a fluid by using current flow parameters of the fluid.

A determination appliance for determining a viscosity of a fluid is presented. The determination appliance has at least one determination device and a provisioning device. The determination device is designed to determine the viscosity of the fluid by using at least one detected volume flow of the fluid and a detected pressure difference of the fluid and/or a rotational speed of a blade wheel for conveying the fluid. The provisioning device is designed to provide or send a viscosity signal that represents the viscosity determined by the determination device.

The determination device can be designed to determine the viscosity by using a functional relationship between the volume flow and the pressure difference to the viscosity and/or by using a lookup table, wherein a relationship between the volume flow and the pressure difference to the viscosity can in particular be stored in the lookup table. Thus, by using the detected volume flow and the detected pressure difference, a viscosity assigned to these values can be read quickly and easily from the lookup table. Or, by using the detected volume flow and the detected pressure difference, the viscosity can be determined quickly and easily by solving the functional relationship. In order to create the lookup table, a calibration of a measurement can, for example, be carried out or may have been carried out in advance such that both the viscosity varies in the relevant range and a rotational speed of, for example, a pump device for conveying the fluid varies in the relevant range, and the resulting pump flows are or were measured. Alternatively or additionally, on the basis of the calibration values, an empirical function, for example, can be determined or may have been determined, with the help of which the viscosity can subsequently be calculated. The lookup table and/or the functional relationship can be stored in the determination device or can be read by the determination device for use.

The determination device can, for example, also be arranged outside the body of a patient in order to determine the viscosity of the fluid data such as the aforementioned detected volume flow of the fluid and a detected pressure difference of the fluid and/or a rotational speed of a pump. For this purpose, the determination device can then, for example, obtain the values or parameters that are required for the determination of the viscosity wirelessly or via a signal line so that it can also determine the viscosity of the fluid outside the body of the patient.

The determination appliance can have a cannula with a receiving interface for receiving the fluid and a discharge interface opposite the receiving interface for discharging the fluid, wherein the pressure difference can in particular represent a difference between a pressure of the fluid in the region of the receiving interface and a further pressure of the fluid in the region of the discharge interface and/or the volume flow can represent a volume flow of the fluid through the cannula. Such a cannula can be formed for use on or in a heart support system. For example, the cannula can be formed or designed to receive blood as the fluid. The current viscosity of the blood in the cannula can thus be advantageously determined by using the determination appliance.

It is furthermore advantageous when the determination appliance has, according to one embodiment, a blade wheel for conveying the fluid from the receiving interface to the discharge interface of the cannula, wherein the blade wheel is arranged or can be arranged in particular on or in the region of the discharge interface. The blade wheel can be arranged, for example, in a discharge section adjacent to the discharge interface. The volume flow of the fluid and the pressure difference can thus be effected during operation of the blade wheel.

In this case, it is advantageous for the determination appliance to have a volume flow sensor, which is designed to detect a volume flow of the fluid through the cannula and to provide or send it to the determination device, wherein the volume flow sensor is in particular arranged in the region of the receiving interface. A current volume flow can thus be taken into account to determine the viscosity.

This volume flow sensor can have at least one Doppler sensor for detecting Doppler ultrasound and/or a thermofilament anemometry sensor and/or an optical sensor. The thermofilament anemometry sensor can have a sensor element, e.g. a wire, wherein the sensor element can be electrically heated and its electrical resistance is a function of the temperature. A heat transfer into the fluid can take place as a result of the flow pattern, said heat transfer changing with the flow velocity. By measuring the electrical variables, the flow velocity can thus be deduced.

The determination appliance can also have a pressure sensor device with at least one differential pressure sensor and/or two barometric pressure sensors, wherein the pressure sensor device can in particular be designed to detect a pressure difference between two sensor points on two opposite sides of the blade wheel and to provide or send it to the determination device. A current pressure difference can thus be taken into account to determine the viscosity.

It is furthermore advantageous for the determination appliance to have a drive device, coupled or couplable to the blade wheel, for driving the blade wheel, wherein the determination device can in particular be designed to determine the viscosity by using a drive parameter of the drive device and/or of the blade wheel. In this case, the determination device can be designed to determine the viscosity by using a drive parameter of the drive device and/or of the blade wheel during operation of the drive device and/or of the blade wheel. The drive parameter can be understood to be an electrical power consumption of the drive device and/or a rotational speed and/or an angular speed of the blade wheel. Such a determination appliance can be formed or usable as a heart support system. This heart support system can advantageously determine a current blood viscosity and, for example, provide or send it for a diagnostic method.

Furthermore, a method for determining a viscosity of a fluid is presented. The method has a step of determining and a step of providing. In the step of determining, the viscosity of the fluid is determined by using at least one detected volume flow of the fluid and a detected pressure difference of the fluid. In the step of providing, a viscosity signal representing the viscosity determined in the step of determining is provided or sent.

This method can be carried out by using the previously presented determination appliance. The method can, for example, be implemented in software or hardware or in a mixed form of software and hardware in a control device, for example.

Advantageous is also a computer program product or computer program with program code which can be stored in a machine-readable carrier or storage medium, such as a semiconductor memory, a hard drive memory, or an optical memory, and is used to carry out, implement, and/or control the steps of the method, in particular when the program product or program is executed on a computer or an appliance.

Exemplary embodiments of the approach presented here are shown in the drawings and explained in more detail in the following description. The figures show:

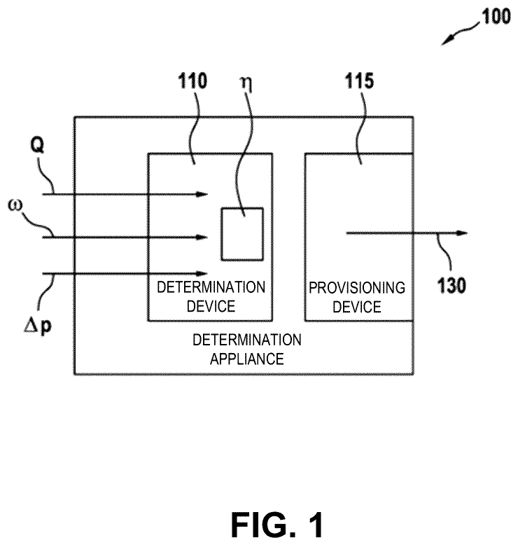

FIG. 1 a schematic illustration of a determination appliance for determining a viscosity of a fluid according to an exemplary embodiment;

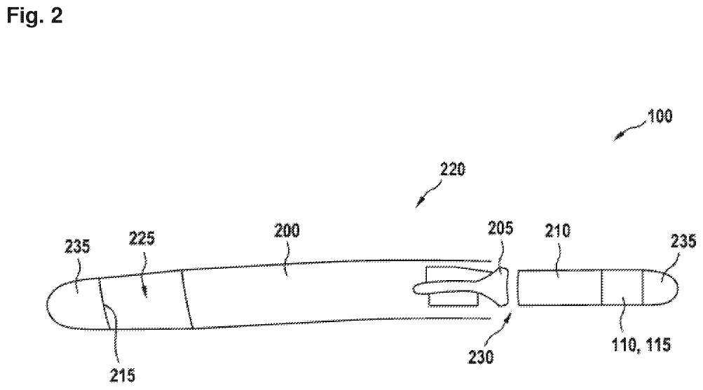

FIG. 2 a schematic side view of a determination appliance according to an exemplary embodiment;

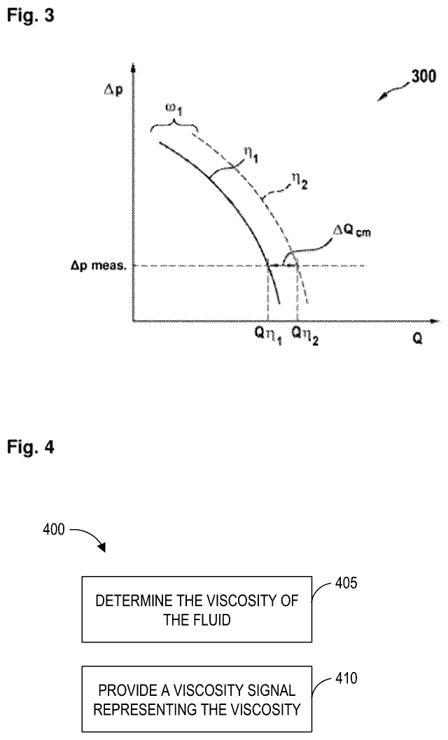

FIG. 3 a characteristic map of pressure difference over volume flow for various viscosities for use with a determination appliance according to an exemplary embodiment; and

FIG. 4 a flow diagram of a method for determining a viscosity of a fluid according to an exemplary embodiment.

In the following description of favorable exemplary embodiments of the present approach, the same or similar reference signs are used for the elements that are shown in the various figures and have a similar effect, wherein a repeated description of these elements is omitted.

If an exemplary embodiment includes an “and/or” conjunction between a first feature and a second feature, this should be read to mean that the exemplary embodiment according to one embodiment comprises both the first feature and the second feature and according to another embodiment comprises either only the first feature or only the second feature.

FIG. 1 shows a schematic illustration of a determination appliance 100 for determining a viscosity η of a fluid according to an exemplary embodiment.

The determination appliance 100 has a determination device 110 and a provisioning device 15. The determination device 110 is designed to determine the viscosity η of the fluid by using at least one detected volume flow Q of the fluid and a detected pressure difference Δp of the fluid. The provisioning device 115 is designed to provide or send a viscosity signal 130 representing the viscosity η determined by the determination device 110. According to this exemplary embodiment, the determination device 110 is designed to read the detected volume flow Q and the detected pressure difference Δp in the form of sensor signals.

FIG. 2 shows a schematic side view of a determination appliance 100 according to an exemplary embodiment. This determination appliance can be the determination appliance 100 described with reference to FIG. 1, with the difference that the determination appliance 100 according to this exemplary embodiment additionally has a cannula 200, a blade wheel 205, a drive device 210, a volume flow sensor 215, and a pressure sensor device 220.

The determination appliance or a determination device 110 can be integrated into the pump or arranged outside the body of a patient when no or only little installation space is available for microelectronic elements for measuring parameters that are required to determine the viscosity. In this case, for example, the electronics or corresponding components of the determination device 110 can be accommodated in a remote (implanted) control device so that predominantly a pressure sensor and/or a transducer element of the volume flow sensor can then be accommodated in the pump itself or implanted in the patient. The sensor values of the corresponding sensor(s) implanted in the patient can then be transmitted out of the patient wirelessly or by means of a signal line and processed in the determination device 110, for example, in a bag or on a belt of the patient, in order to determine the viscosity of the fluid (in this case, blood). Such an exemplary embodiment is not explicitly shown in the figures appended here.

Additionally or alternatively, the viscosity of the fluid can of course also be determined by means of a determination device 110 in the form of a cloud server, so that in this case, the sensor values required for the determination are to be transmitted via the internet or a corresponding signal line. An appropriate protection or encryption of this data against unauthorized tapping or reading of this data by unauthorized persons should advantageously be ensured in this case.

An arrangement of the determination device 110 in three options is thus conceivable:
1) Calculation of the viscosity by a determination device 110 implanted in the patient (e.g. as a control device or also in a pump, especially when corresponding hardware components have sufficiently small dimensions)
2) Extracorporeal calculation of the viscosity, e.g. close to the body of the patient (e.g. in a box attached to a belt of the patient)
3) Calculation of the viscosity further away from the patient (e.g. in a non-portable component, such as a tabletop device, an analysis device in a medical practice, or even a cloud server).

The cannula 200 has a receiving interface 225 formed to receive the fluid and a discharge interface 230 opposite the receiving interface 225 formed to discharge the fluid.

The blade wheel 205 is designed to convey the fluid from the receiving interface 225 to the discharge interface 230 of the cannula 200. According to this exemplary embodiment, the blade wheel 205 is arranged in the region of the discharge interface 230 and/or in the cannula 200.

The volume flow sensor 215 is designed to detect a volume flow of the fluid through the cannula 200 and to provide or send it to the determination device 110. Accordingly, the volume flow represents a volume flow of the fluid through the cannula 200. According to this embodiment example, the volume flow sensor 215 is arranged in the region of the receiving interface 225 for this purpose. According to this exemplary embodiment, the volume flow sensor 215 has a Doppler sensor. According to an alternative exemplary embodiment, the volume flow sensor 215 additionally or alternatively has a thermofilament anemometry sensor and/or an optical sensor.

According to this exemplary embodiment, the pressure sensor device 220 has two barometric pressure sensors 235, which are designed to detect a pressure difference between two sensor points on two opposite sides of the blade wheel 205 and to provide or send it to the determination device 110. According to an alternative exemplary embodiment, the pressure sensor device 220 additionally or alternatively has at least one differential pressure sensor. According to this exemplary embodiment, the pressure sensors 235 are arranged in the region of the receiving interface 225 and in the region of the discharge interface 230. Accordingly, the pressure difference according to this exemplary embodiment represents a difference between a pressure of the fluid in the region of the receiving interface 225 and a further pressure of the fluid in the region of the discharge interface 230.

The drive device 210 is coupled to the blade wheel 205 and designed to drive the blade wheel 205. According to this exemplary embodiment, the determination device 110 is designed to determine the viscosity by using a drive parameter of the drive device 210 and/or of the blade wheel 205 during operation of the drive device 210 and/or of the blade wheel 205.

In this case, the determination device 110 can, for example, be arranged outside the patient or a pump, e.g. in a portable control device. Sensor values of sensors implanted in the patient can then, for example, be supplied wirelessly or by means of a signal line to the determination device 110.

In the following, details of the determination appliance 100 are described again in more detail and in other words:

According to this exemplary embodiment, the determination appliance 100 presented here can be used as a heart support system. For patients with a heart support system, also called VAD patients (VAD stands for “Ventricular Assist Device”), coagulation management is essential for minimizing pump thromboses. For this purpose, patients are, for example, treated with medications to inhibit plasmatic blood coagulation, and the INR is adjusted in the range of 2 to 2.5, for example.

A mechanical load on the drive device 210 of a VAD system, i.e. of a heart support system, is a function of the volume flow, the pressure difference, and the viscosity. With a known volume flow and known pressure difference, which are measured in the determination appliance 100 presented here via sensors 215, 220, the viscosity of the fluid, in this case blood, can be deduced from the electrical power consumption of the drive device 210. For this purpose, the determination device 110 according to this exemplary embodiment is designed to read as the drive parameter a parameter which represents or makes it possible to determine the mechanical load on the drive device 210 and/or on the blade wheel 205. In this case, the determination device 110 is advantageously designed to divide the power consumption of the pump, consisting of the drive device 210 and the blade wheel 205, into a volume flow contribution and a viscosity contribution. The flow measurement is realized on the basis of ultrasound according to this exemplary embodiment or anemometrically according to an alternative exemplary embodiment. A direct determination of the viscosity during the operation of the determination appliance 100 is advantageously possible in this case by means of an explicit Doppler ultrasonic volume flow measurement. Advantageously, a pumping performance of the pump does not have to be interrupted for this purpose.

The blood viscosity is determined during operation of the determination appliance 100 by the determination device 110 continuously according to this exemplary embodiment or in fixed time intervals according to an alternative exemplary embodiment. The provisioning device 115 is designed to provide the determined viscosity to a physician and/or patient as a parameter for carrying out the treatment. For this purpose, the viscosity signal is designed to display the viscosity on a display and/or transmit it by radio transmission to a web service. As already stated above, the determination device 110 can also be arranged outside the patient, e.g. in a bag that the patient carries along. Signal values of sensors implanted in the patient can then, for example, be transmitted to the determination appliance wirelessly and/or by means of a signal line.

A determination appliance 100 presented here contains a system consisting of a pump drive in the form of the drive device 210, the blade wheel 205, and the cannula 200 also called the inlet cannula, the volume flow sensor 215 for measuring the pump volume flow actually conveyed by the drive and the blade wheel 205, in this case, by means of Doppler ultrasound, optionally or additionally by means of thermofilament anemometry and/or optical methods. An integration of the volume flow sensor 215 in the form of a Doppler ultrasonic sensor in a tip of the inlet cannula is shown here. In addition, the determination appliance 100 comprises two barometric pressure sensors 235 for forming the pressure difference in the determination device 110, which, according to this exemplary embodiment, has a data processing device in the form of a microcontroller. According to an alternative exemplary embodiment, the determination appliance 100 has at least one differential pressure sensor for determining a pressure gradient across the blade wheel 205 in the form of an impeller.

Calculation examples follow for illustrating possible methods of the determination device 110 when determining the viscosity, see also FIG. 3 in this respect:

The hydraulic power of the pump P_{hydraulic} is a function of an angular speed ω, a hydraulic efficiency η_{hydraulic}, and a load torque M, wherein the load torque M is a function of the viscosity.

This relationship can be represented in the following equation:

$${P}_{hydraulic} = ω \cdot M \cdot {η}_{hydraulic}$$

The hydraulic performance P_{hydraulic} is also a function of the pressure difference Δp and the volume flow Q or volumetric flow. This relationship can be represented in the following equation:

$${P}_{hydraulic} = Δρ \cdot {Q}_{}$$

If the pump is now operated at a defined angular speed ω1 and the actual volume flow Q_{ω1} is measured, the viscosity η can be determined, as illustrated in FIG. 3, from this measured volume flow Q_{ω1}, according to this exemplary embodiment by means of Doppler ultrasonic sensors. For this purpose, according to this exemplary embodiment, a calibration of the measurement was carried out in advance such that both the viscosity varies in the relevant range and the rotational speed varies in the relevant range, and the resulting pump flows were measured. From this, “lookup tables,” or LUT for short, were then created, with the help of which a viscosity can then be assigned by the determination device 110 to a measured pressure difference and a measured volume flow at a given rotational speed/angular speed. This measurement and calibration was carried out according to this exemplary embodiment by using the determination appliance 100. According to an alternative exemplary embodiment, the viscosity is determined by the determination device 110 by determining on the basis of the calibration values a functional relationship in the form of an empirical function, with the help of which the viscosity can subsequently be calculated:

η=f(Δp, Q, ω)

As an alternative to the use of the angular speed ω, the electrical power consumption is used for the calculation by the determination device 110 according to an alternative exemplary embodiment, because:

$${P}_{el} = {P}_{hyd} / ( {η}_{el} \cdot {η}_{mech} \cdot {η}_{hyd} )$$

This was determined in preliminary tests by measuring the torque and the rotational speed as well as the voltage U and current I by using the determination appliance 100. Under the premise that the further mechanical losses are only a function of the rotational speed and pressure, which is true in a very good approximation in the case of one of the determination appliances 100 presented here, it can be assumed that η_{mech} is constant and therefore does not play a role in the viscosity determination.

The volume flow sensor 215 can be proven optically or by fluoroscopy. The calculation of the viscosity from the pressure difference, volume flow, and/or angular speed can be proven in a purposeful experiment by manipulating the volume flow or the pressure difference.

FIG. 3 shows a characteristic map 300 of pressure difference Δp over volume flow Q for various viscosities η for use with a determination appliance according to an exemplary embodiment. This determination appliance can be one of the determination appliances 100 described in FIG. 1 or 2. According to this exemplary embodiment, the characteristic map 300 is stored in the form of the lookup table described in FIG. 2 or the functional relationship in the determination device of the determination appliance or can be read by the determination device.

For Δpₘₑₐₛ=¢& ω=¢, it follows that

Q=f(η) where Q_{η2}>Q_{η1} for η₁>η₂

FIG. 4 shows a flow diagram of a method 400 for determining a viscosity of a fluid according to an exemplary embodiment. This method can be a method 400 that can be carried out or controlled by one of the determination appliances described with reference to one of FIG. 1 or 2.

The method 400 has a step 405 of determining and a step 410 of providing. In step 405 of determining, the viscosity of the fluid is determined by using at least one detected volume flow 10 of the fluid and a detected pressure difference of the fluid and/or a rotational speed of a blade wheel for conveying the fluid. In the step 410 of providing, a viscosity signal representing the viscosity determined in the step 405 of determining is provided or sent.

The method steps presented here can be repeated as well as carried out in a sequence other than the one described.

1-11. (canceled)

12. A ventricular assist system comprising:
an impeller;
a cannula configured to be implanted in a body of a patient, wherein the cannula comprises:
a lateral receiving interface distal to the impeller, configured to receive blood of the patient into an interior of the cannula; and
an outlet interface, wherein the impeller is configured to convey the blood from the lateral receiving interface to the outlet interface; and
a volumetric flow sensor positioned at a tip of the cannula distal to the impeller and configured to sense a volumetric flow (Q) of the blood of the patient through the cannula, the volumetric flow sensor comprising a Doppler ultrasonic sensor.

13. The ventricular assist system of claim 12, further comprising a determination device configured to determine a viscosity (η) of blood in the body of the patient based on at least the sensed volumetric flow (Q) of the blood, a pressure difference (Δp) of the blood in a region of the receiving interface and a region of the outlet interface, and either a rotational speed (ω) of the impeller or an electric power input (Pₑ₁) of a drive device for the impeller.

14. The ventricular assist system of claim 13, wherein the determination device is configured to determine the viscosity (η) based on a functional relationship between the volumetric flow (Q), the pressure difference (Δp), and the viscosity (η).

15. The ventricular assist system of claim 13, wherein the determination device is configured to determine the viscosity (n) using a lookup table.

16. The ventricular assist system of claim 15, wherein a relationship between the volumetric flow (Q), the pressure difference (Δp), and the viscosity (η) is stored in the lookup table.

17. The ventricular assist system of claim 13, further comprising a pressure sensor device, wherein the pressure sensor device comprises at least one differential pressure sensor or two barometric pressure sensors, wherein the at least one differential pressure sensor or two barometric pressure sensors are configured to measure the pressure difference (Δp) between two sensor points.

18. The ventricular assist system of claim 13, further comprising a pressure sensor device configured to sense the pressure difference (Δp) between two sensor points.

19. The ventricular assist system of claim 12, wherein the impeller is positioned in a region of the outlet interface.

20. A method for determining a viscosity (η) of blood of a patient in a ventricular assist system, comprising:
sensing a volumetric flow (Q) using a volumetric flow sensor of the ventricular assist system, the volumetric flow sensor comprising a Doppler ultrasonic sensor arranged in a distal tip of a cannula of the ventricular assist system, the cannula, configured to be implanted in a body of the patient, wherein the ventricular assist system comprises:
an impeller configured to convey blood from a lateral receiving interface of the cannula distal to the impeller to an outlet interface of the cannula.

21. The method of claim 20, further comprising determining a viscosity (η) of the blood based on at least the sensed volumetric flow (Q) of the blood, a pressure difference (Δp) of the blood in a region of the receiving interface and a region of the outlet interface, and either a rotational speed (@) of the impeller or an electric power input (Pₑ₁) of a drive device for the impeller.

22. The method of claim 21, wherein determining the viscosity (η) comprises determining the viscosity (η) based on a functional relationship between the volumetric flow (Q), the pressure difference (Δp), and the viscosity (η).

23. The method of claim 21, wherein determining the viscosity (η) comprises using a lookup table.

24. The method of claim 23, wherein a relationship between the volumetric flow (Q), the pressure difference (Δp), and the viscosity (η) is stored in the lookup table.

25. The method of claim 21, further comprising determining the pressure difference (Δp) using a pressure sensor device comprising at least one differential pressure sensor or two barometric pressure sensors.

26. The method of claim 21, further comprising determining the pressure difference (Δp) using a pressure sensor device configured to sense the pressure difference (Δp) between two sensor points.

27. The method of claim 20, wherein the impeller is positioned in a region of the outlet interface.

28. A non-transitory computer-readable storage medium comprising instructions that, when executed, direct a processor to perform a method comprising:
sensing a volumetric flow (Q) using a volumetric flow sensor of a ventricular assist system, the volumetric flow sensor comprising a Doppler ultrasonic sensor arranged in a distal tip of a cannula of the ventricular assist system, the cannula, configured to be implanted in a body of a patient, wherein the ventricular assist system comprises:
an impeller configured to convey blood from a lateral receiving interface of the cannula that is distal to the impeller to an outlet interface of the cannula.

29. The non-transitory computer-readable storage medium of claim 28, wherein the method further comprises determining a viscosity (η) of the blood based on at least the sensed volumetric flow (Q) of the blood, a pressure difference (Δp) of the blood in a region of the receiving interface and a region of the outlet interface, and either a rotational speed (ω) of the impeller or an electric power input (Pₑ₁) of a drive device for the impeller.

30. The non-transitory computer-readable storage medium of claim 29, wherein determining the viscosity (η) comprises determining the viscosity (η) based on a functional relationship between the volumetric flow (Q), the pressure difference (Δp), and the viscosity (η).

31. The non-transitory computer-readable storage medium of claim 29, wherein determining the viscosity (η) comprises using a lookup table.
